(19) United States
(12) Patent Application Publication
(10) Pub. No.: US 2019/0189257 A1
(43) Pub. Date: Jun. 20, 2019

(54) METHOD AND SYSTEM FOR OPIOID DEPENDENCY/ADDICTION TREATMENT

(71) Applicant: Kevin ROD, Toronto (CA)

(72) Inventor: Kevin ROD, Toronto (CA)

(21) Appl. No.: 15/843,940

(22) Filed: Dec. 15, 2017

(51) Int. Cl. G16H 20/10; G16H 80/00; G16H 10/20; G09B 19/00; G09B 7/10; G09B 5/06; A61K 36/185; G16H 10/60; G16H 20/70

(52) U.S. Cl. CPC G16H 20/10; G16H 80/00; G16H 10/20; G09B 19/00; G16H 20/70; G09B 5/065; A61K 36/185; G16H 10/60; G09B 7/10

(57) ABSTRACT

The present invention is a method for reducing addiction to opioids. The method comprises of a pharmacological treatment with assistance of a psychological treatment program. The pharmacological treatment advocates opioid reduction with assistance of proper use of medical cannabis by 1-3 g per day (maximum 5 g per day) which reduces opioids by increments of 10%. In opioid reduction plan of the present invention the opioid is gradually replaced with medical cannabis. The present invention provides a psychological treatment program using an online platform/app/website for assessment of patients and provides solution and helps with an abnormal condition during the opioid reduction treatment.

### FIELD OF THE INVENTION

The present invention relates in general to methods for reducing dependency/addiction to opioids in patients experiencing opioid treatment for pain and in particular to a method for reducing opioid dependency/addiction by providing a cannabis treatment with assistance of a psychological support platform called Medical Cannabis Opioid Reduction Program: MCORP.

### BACKGROUND OF THE INVENTION

Patients who are experiencing severe pain may be prescribed with opioid drugs. Opioids like codeine, oxycodone, morphine or Fentanyl are very effective treatments for pain. Prolong use of many opioids (especially at high doses) carries a significant risk of dependency/addiction. The physical dependence on such drugs may also lead to a psychological dependence, when even the thought of going without the medication may trigger intense anxiety.

The majority of existing methods for treating opioid addiction provide only a pharmacological support. They usually use other drugs to reduce opioid use. Medical Cannabis is one of the known pharmacological treatments for opioid reduction. Research has shown that there are potential benefits to medical cannabis including but not limited to: pain relief, such as chronic pain or cancer related pain, neuropathic pain; pain in patients with HIV/AIDS and in patients receiving chemotherapy; autoimmune disease such as multiple sclerosis; epilepsy; glaucoma; neurodegenerative disease and post-traumatic stress disorder (PTSD). The use of medical cannabis as a replacement for opioid medications, or to get off of opioids or cut back, is proven to be effective. The medical cannabis helps in replacing opioids as painkillers and increases the effects of opioids, leading users to take lower doses for the same level of relief.

The plant Cannabis sativa has been used in medicinal practice for thousands of years. The pharmacologically active constituents of the plant are termed cannabinoids. In some aspects, the cannabis plant material is derived from cannabis strains having different levels of THC and CBD. Cannabis plants are categorized based on the overall amount of THC produced, and on the ratio of THC to CBD. Some Cannabis strains have been bred to produce minimal levels of THC, the principal psychoactive constituent responsible for the high associated with it and other strains have been selectively bred to produce high levels of THC and other psychoactive cannabinoids.

Psychological support is also shown to be highly effective in opioid dependency/addiction treatment. However, these types of supports are expensive and out of reach of many patients. Therefore, only a few patients on such drugs use such supports. Less than ten percent of the people that suffer with pain obtain psychotherapy.

Considering the current state of Opioid Use Disorder in many countries, there is a need for a novel method to reduce opioid dependency/addiction and support the patient in a daily program to bring them to a healthier life. The present invention is a secure and self-help method of opioid reduction using both cannabis and psychological treatment to help users to have a successful treatment process and maintain a well-balanced life through their daily practices.

### SUMMARY OF THE INVENTION

The present invention relates in general to a method and a system for reducing dependency/addiction to opioids in patients. The method comprises of a combined pharmacological and psychological treatment program. The system provides a unique reduction plan based on each patient's response to the opioid reduction.

In the present opioid reduction plan, the opioid is gradually replaced with medical cannabis so that the amount of cannabinoids administered is increased week by week or as indicated until a certain saturation point, which is determined based on response, weight, and monthly-quarterly test results, is reached. Based on the present opioid dependency/addiction treatment method the total pharmacological cannabis consumed daily is: 0.5-3 g per day (maximum 5 g per day), which reduces opioids by increments of 10%. The dosage varies with patient and route of administration. The pharmacological system of the present invention recommends up to 500 mg medical cannabis for each 10% reduction in opioid dose if tolerated and indicated. This recommended medication can be consumed by Buccal, Oral, Vaping or other methods.

The pharmacological treatment is supported by a psychological support (trade mark named ZENDOSE). The present invention provides a psychological treatment program using a unique online platform/app/website for assessment of patients and based on their needs, send them solutions and based on their progress adjusts what further material is to be sent to them to either help them with an abnormal condition or help them maintain their wellness once they are going through the opioid reduction treatment.

The system uses an online platform for storing information corresponding to each patient. The system can also use online interactive, educational treatments. The psychological treatment of the system provides daily motivative and encouraging posts to support patient in abnormal conditions, daily exercises in 5 areas: physical, behavioral, mental, emotional, self and interpersonal. Provides assessment to determine issues in 6 areas, depression, anxiety, substance abuse, sleep, physical wellbeing and nutrition. The system will monitor patient, for pain, symptoms, patient's struggles, mood and function and provide solutions.

The systems provides an opioid addiction reduction plan by maintaining a reduction plan for a patient with pain, receive information associated with the patient, and generate a reduction plan based on patient information to assist the patient in managing the reduction plan.

The psychological support encourages the patient step by step in a manner that patients may more efficiently, effectively, and/or consistently manage their medical conditions compared with conventional techniques. This enables the patient to lead a healthy and productive life, and/or reduce the overall cost of health care for everyone.

It is one object of the present system to help patients to maintain a well-balanced life while undergoing an opioid reduction treatment through daily practices, and to improve and maintain their quality of life.

It is another object of the present invention to provide a combination of a pharmacological and psychological method and system for opioid reduction in patients with pain.

It is another objective of the present invention to provide an online method and system for opioid reduction in patients suffering with pain in a convenient, private, and self-help manner.

It is another objective of the present invention to provide a method and system that includes interactive, educational treatment solution based on cognitive behavioral therapies to help patients in a motivative manner and monitor their progress with regular monitoring.

It is another object of the present invention to provide a method and system through daily practice in a fun and engaging manner, so that the patient not only receives opioid reduction treatment but also work on life challenges, as well as ways to improve and maintain their quality of life.

It is another objective of the present invention to provide electronic treatment which is available any day and any time.

It is another objective of the present invention to provide a method and system for opioid reduction that allows for information to be gathered from patients that can be used to assess treatment effectiveness from session to session. This data collection allows for future advancements in individualized and customized treatments and research.

It is another objective of the present invention to provide a method and system, which can be used in any therapy management or mental health/wellness system for providing effective healthy lifestyle.

It is another objective of the present invention to provide a treatment system which uses less therapist time and fewer resources, with the same as or better outcomes than traditional face-to-face therapy.

It is another object of the present invention to provide a safe treatment that can relieve the patients with any type of pain and from any source, including acute pain, chronic pain, nociceptive pain, neuropathic pain, pains caused by surgery, diabetes, trigeminal neuralgia, fibromyalgia, cancer, central pain syndrome, tissue damage, physical injury, and pains with unknown sources.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1 is a flow chart illustrating an opioid reduction treatment method and system of the present invention;

FIG. 2 is a flow chart illustrating questionnaire and feedback process for an embodiment of the present invention;

FIG. 3 is a flow chart illustrating pharmacological treatment of opioid reduction method of the present invention;

FIG. 4 illustrates a system environment in which the features and principles of the present invention implemented;

FIG. 5 is a flow chart illustrating psychological treatment of opioid reduction method of the present invention;

FIG. 6 is a flow chart illustrating psychological treatment platform of opioid reduction method of the present invention;

FIG. 7A illustrates exemplary interfaces according to the principles of the present invention;

FIG. 7B illustrates exemplary interfaces according to the principles of the present invention;

FIG. 7C illustrates exemplary interfaces according to the principles of the present invention, and

FIG. 7D illustrates exemplary interfaces according to the principles of the present invention.

### DETAILED DESCRIPTION OF PREFERRED EMBODIMENTS

The figures are not intended to be exhaustive or to limit the present invention to the precise form disclosed. It should be understood that the invention can be practiced with modification and alteration, and that the disclosed technology be limited only by the claims and equivalents thereof.

The invention disclosed herein, in accordance with one or more various embodiments, is described in detail with reference to the following figures. The drawings are provided for purposes of illustration only and merely depict typical or example embodiments of the disclosed invention. These drawings are provided to facilitate the reader's understanding of the disclosed invention and shall not be considered limiting of the breadth, scope, or applicability thereof. It should be noted that for clarity and ease of illustration these drawings are not necessarily made to scale.

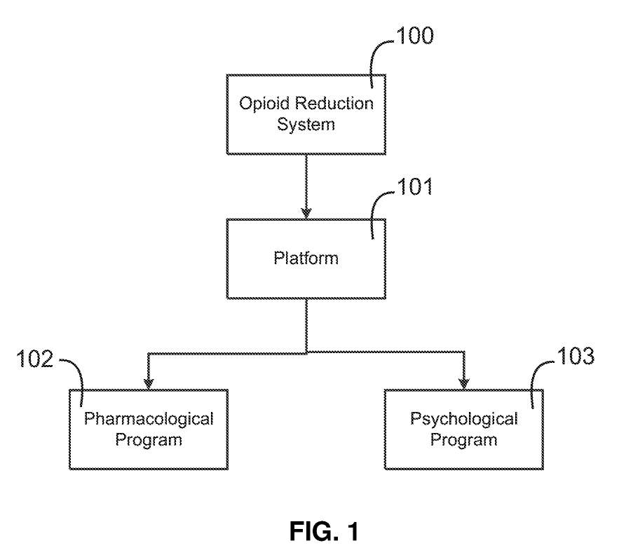

FIG. 1 is a block diagram of the opioid reduction treatment system 100 showing the main aspects of the present invention. The system 100 comprises an opioid reduction platform 101 upon which the system identifies eligible patients who are ready to reduce their opioid doses, regularly assesses patients throughout the reduction program, adapts the program to their individual needs and follows up throughout their reduction procedure. The system 100 designs an individualized and customized opioid reduction program with both pharmacological 102 and psychological 103 supports for the patients who are on opioids for reducing their pain.

The pharmacological program 102 is a medical opioid reduction system with the assistance of proper use of a medical cannabis and the psychological program 103 is a method based on Cognitive Behaviour Therapy, Mindfulness training (Self-management, proven to be effective for pain, depression, anxiety). The psychological program designs an individualized opioid reduction program. The most common causes of relapse during opioid reduction is depression due to high pain scores and negative thinking. The strategy in this case is providing motivational support.

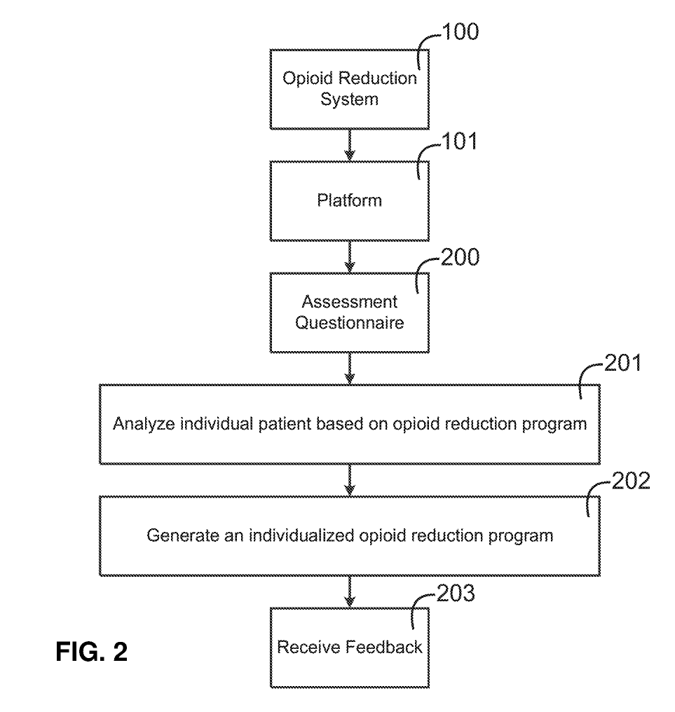

According to FIG. 2 the platform 101 is able to assess the patients through a questionnaire 200. Through a questionnaire the system will identify and analyse each patient 201. The questions are developed based on the specific strategy of the reduction system by the expert physicians, pharmacists and psychologists to analyse the medical history and physiological and psychological stages of the patient. The questionnaire discovers:
patients who are ready to reduce their opioid doses;
suitable patients;
the reasons of a patient's desire to reduce the opioid doses;
overt addiction or dependency;
if any patient has authorized medical cannabis for chronic pain, and
If a patient have observed any side effects, such as, decreased concentration and memory, drowsiness, changes in mood, constipation, dry mouth, abdominal pain, nausea, diminishing analgesia, reduced function and quality of life, absence of progress toward functional goals, etc.

The system will then generate an individualized opioid reduction program for patient based on the targeted program 202. The program will be entered in the computer system, the results of which will be then be monitored in the system. The system will provide feedbacks and monitor each patient 203.

The opioid reduction platform 101 includes a storage facility to maintain reduction plan data representative of set of guidelines, to manage, treat, or otherwise care for a patient in opioid dependency/addiction reduction plan. For example, it may specify one or more exercise, provide one or more notification for a particular condition that the patient should perform in order to manage the specific condition.

In an embodiment the opioid addiction reduction system 100 may include a general plan that may be used by a plurality of patients that have the same medical condition and are using the same dosage of a particular opioid. In this case the system provides the same pharmacological plan. Alternatively, the system may be personalized to a particular patient. The system receives input provided by a health care practitioner personalized to a particular patient.

The system may automatically initiate a consultation between the patient and the health care practitioner. In some examples, the consultation may include a virtual consultation by way of the patient portal and the practitioner portal. Additionally or alternatively, the health care practitioner may specify one or more practice that may be performed by the patient in order to improve a health characteristic associated with the medical condition.

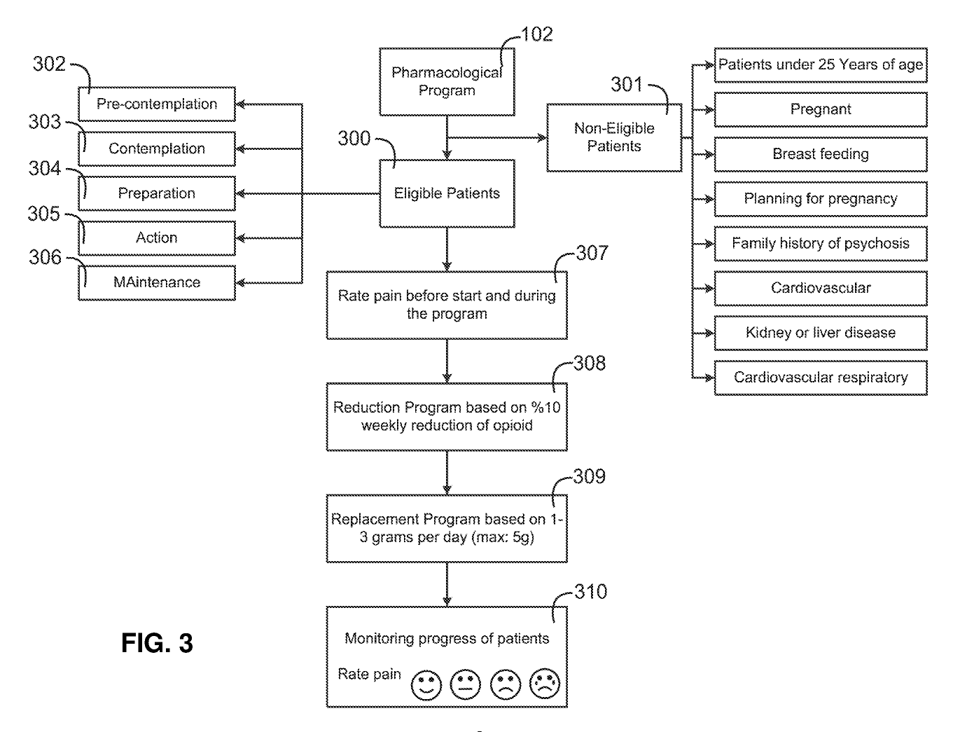

According to FIG. 3, once the questionnaire is completed, the results of the questionnaire are received by the system and a pharmacological program 102 is developed for eligible patients 300. Patients under 25 years of age, patients with personal/strong family history of psychosis, Unstable or bipolar mood aisorders, pregnant, breast-feeding or of planning a pregnancy, active substance use disorder, cardiovascular, respiratory, severe kidney or severe liver disease are considered as inappropriate candidates 301.

The system divides the patients into 5 categories based on their opioid reduction program:
1—Pre-contemplation 302: In this category, a patient is not thinking of reduction. The strategy of the system is to arrange motivational programs.
2—Contemplation 303: In this category, a patient is considering potential benefits of reduction. The strategy in this category is preparing motivational program.
3—Preparation 304: In this category, a patient is ready to begin reduction, but unsure. The strategy in this case is to set small, achievable goals to build confidence and provide support.
4—Action 305: In this category, a patient is committed to reduction. The strategy is ongoing monitoring and support.
5—Maintenance 306: In this category, a patient may lose motivation over time. The strategy is to provide motivative and encouraging tips.

The pharmacological part 102 of the opioid reduction system uses the medical cannabis to support patients through opioid reduction with a view to discontinuation. Medicinal grade plants produce relatively low levels of THC and high levels of CBD, while drug plants produce high levels of THC and low levels of CBD. CBD is more analgesic and neuroprotective, while also offering the potential to reduce neuroinflammation and to stimulate neurogenesis. Medical cannabis must be used with caution with patients with concurrent mood/anxiety disorders, tobacco smokers, heavy alcohol drinkers, high risk factors for C-V disease and taking heavy doses sedating medication.

The system comprises a software that contains information regarding dosing schedules for the unit dose for daily usage for each patient based on the information received from the patient. The software program provides a recommended dosing based on the information entered.

The system rates patients pain and function 307 before beginning the program and throughout the reduction and assesses patients and aware patients of the risk of substance misuse. The system monitors the amount of pain of the patient before and after the treatment: pain is evaluated based on pain intensity, pain quality and pain location. The system further identifies the clinical and psychological factors that increase and decrease patients pain; reviews of the effectiveness of any opioids that have been tried to relieve patients pain; reviews the worst, least, and typical pain experienced over the past period of time; psychological factors that impact of the pain on sleep; mood; level of stress; and function in work, relationships, and recreational activities of the patient. The system evaluates the time that patient routinely develops the most pain and provides a time plan for consuming the medical cannabis based on this time.

Through daily posts and educational contents the system alerts patients of acute effects of the medication such as: Perceptual distortions, Cognitive impairment euphoria, Anxiety, Vasodilation, Supine hypertension, Postural hypotension, Heightened sensory perception, Drowsiness, Incoordination, Increased appetite.

According to FIG. 3 again the invention provides method of treating opioid dependency by reducing the amount of opioids used every 2 days to 3 weeks by at least 10%, 308. The maximum daily opioid prescribing dose is equivalent to 90 mg morphine. The reduction dose plan is related to each patient's need. Inversely related to length of opioid therapy 10 percent weekly reduction is usually safe and effective if tolerated. In patients with unstable mental health or cardio-respiratory disease, the reduction plan is slower. A unique reduction plan based on each patient will be provided. The system will consider:
Maintaining same opioid dosing schedule as long as possible.
Extending time between doses at smallest available unit.
Halve rate of reduction at 30 percent original dose.

At all times, the system maintains all other aspects of the multi-model pain control therapy and remains Patient—Centered.

In opioid reduction plan of the present invention, the opioid is gradually replaced with medical cannabis so that the amount of cannabinoids administered is increased week by week until a certain saturation point, which is based on responses of the patient. Total pharmacological cannabis consumed daily is: 0.5-3 g per day (maximum 5 g per day), which may reduce opioids by increments of 10%. Optimal dosage improves function, pain control, quality of life, minimizes euphoria and cognitive impairment. The dosage varies with patient and route of administration. Better to start with low dose 0.5 g per day and maximum 3-5 g per day.

The system provides education about methods of consuming. The patients should be advised to consume in a comfortable environment, wait a few minutes between inhalations or 30-60 minutes after ingesting.

The pharmacological system of the present invention recommends up to 500 mg medical cannabis for each 10% reduction in opioid dose. This recommended medication can be consumed by Buccal, Oral, or Vaping. In inhalation of cannabis, the patient is recommended to vapour over 5 seconds, hold breath for 10 seconds and exhale slowly. Patients should avoid driving for 4 hours after inhaling, 6 hours after ingesting and 8 hours if they experience euphoria. Some of the adverse effects of cannabis are: Potential memory impairment, Reduced performance at work or school, Interference with social relationships.

In stage 310, the system will monitor frequently the pain of patient, effects of reduction program, effects of psychological exercises, benefits of tapering, opioid withdrawal symptoms, renew and adjust the dosage of medication if needed and slow or pause the reduction, if the patient struggles, worsening pain, mood or function, times of intense stress, appearance of withdrawal symptoms.

The system also provides the documents needed for authorizing the use of cannabis for medical purposes under the access to cannabis for medical purposes regulations through qualified health care provider 301. The system also provides list of Licensed producers, Specify the quantity, percent of THC, daily dose and the, period of use

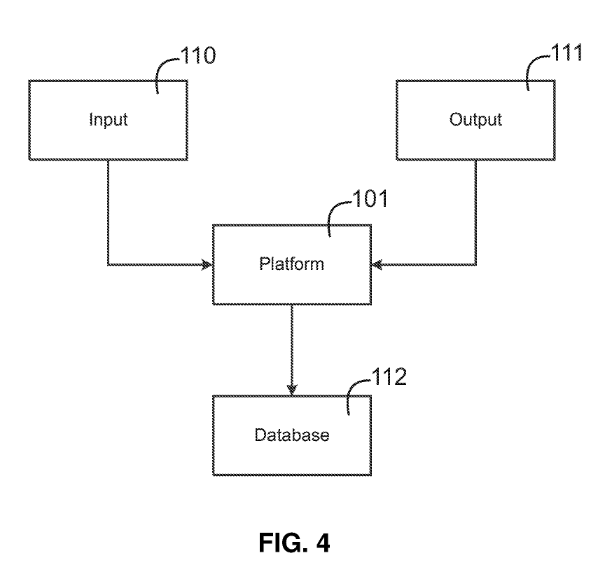

FIG. 4 is a block diagram of platform environment of the system in which the principles of the present invention may be implemented. The system environment consists of an input 110, an output 111, a platform 101 and a database 112. The platform 101 is adapted to include the necessary functionality input through input 110 and access to database 112.

The platform 101 is implemented by a software for carrying-out the features and operations of the system for delivering opioid reduction program and mental health support program customized individually for each patient. For example, the software contains information regarding dosing schedules for the unit dose for daily usage for each patient based on the information received from the patient. The platform 101 may also be provided with a wide variety of components including, for example central processing units, memory, registers, and other data processing devices. The system may include a patient computing device and a health care practitioner computing device, each communicatively coupled to opioid reduction platform system 101 by a network. Patient computing system may be associated with one or more health care practitioners, physicians, nurses, psychologists or therapists.

Patient computing device and health care practitioner computing device may be implemented by a mobile device, a tablet computer, a laptop computer, a personal computer, and/or any other suitable computing device that may be configured to access a patient to reduction program platform. Patient computing device, practitioner computing device, and opioid reduction system 101 may communicate using any communication technologies suitable for transporting data, including known communication devices, media, and protocols supportive of remote or local data communications.

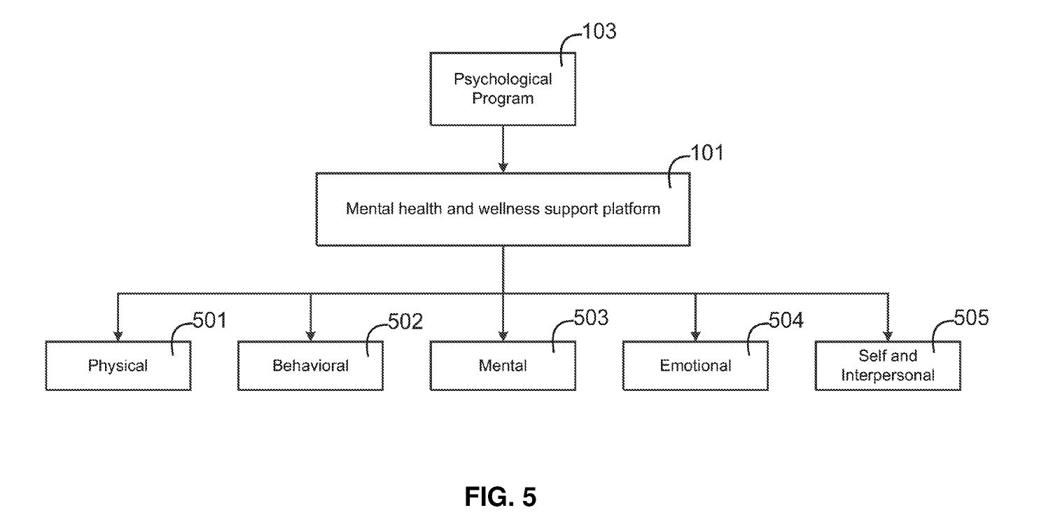

According to FIG. 5, the psychological part 103 is a support platform for a successful opioid reduction program, which provides step by step solutions for problems that may increase throughout the reduction program based on patient's progress and help with abnormal conditions or support to maintain wellness. The opioid reduction platform may automatically select an appropriate notification from the storage of the system based on the input of the patient and the response to input provided by a health care practitioner. For example, opioid reduction platform may receive input provided by the health care practitioner in response to the inputs of the patient and generate a psychological support based on the input provided by the health care practitioner.

The psychological program 103 provides support in 5 areas: Physical 501, behavioural 502, mental 503, emotional 504 and self and interpersonal 505. However, every patient will be assigned at least one aspect in each of the 5 areas to work on. Under physical 501, patients will be assigned to keep track of their sleep or nutrition and get tips for healthy eating habits or both, and complete modules under these headings, exercise and dialectical behavioural therapy. Patients will receive tips for effective meditation, relaxation exercises and breathing exercises. The program guides patients in a fun and engaging manner through daily practice by targeting mild issues and works to find ways to support them through challenges to improve and maintain their quality of life.

Under behavioural 502, patients will learn to utilize techniques to better handle distress, distraction and self-soothing.

Under mental 503, patients will learn about the links between their thoughts and well-being and learn to utilize skills such as mindfulness, monitoring negative self-talk, mindfulness meditation. Changing negative thinking, negative self- talk and cognitive distortion, bringing awareness and change into negative self-talk, evaluating mistaken beliefs, provide daily coping affirmations, awareness without judgement, learn being in the present moment. In this case, the system provides an 8 week challenge mindfulness exercise:
Mindfulness exercise 1: Awareness in everyday activities
Mindfulness exercise 2A: Fully immersed in an activity using the senses
Mindfulness exercise 2B: Awareness of a piece of Toast using the senses
Mindfulness exercise 3: Awareness of time
Mindfulness exercise 4: Thousand thoughts a day
Mindfulness exercise 5: Putting emotions into words
Mindfulness exercise 6: Paying attention to the breath

Under emotional 504, users will learn to identify and deal with difficult emotions, identify emotion and appropriate ways to express feelings and manage anger and frustration.

Under Self and Interpersonal 505, patients will learn about how their relationship with others and themselves can influence their well-being (Assertiveness, relationship building and maintenance, self-esteem and motivation and goal-setting).

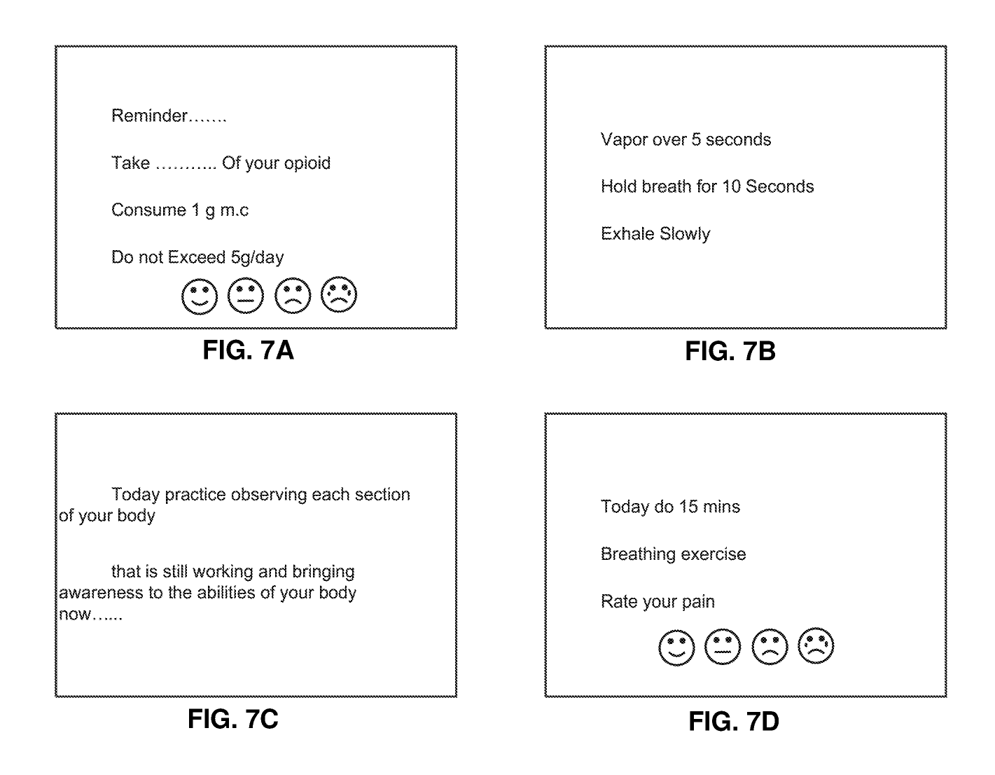

FIG. 7 illustrates the psychological program algorithm from the start when the patient logs in the system and how the information will be presented step by step. The program comprises of 7 steps wherein in each step, the user would complete each of the subsections before moving on the next step. Some steps may need to be repeated as part of daily/weekly exercise.

In step 1, the patient receives a brief description of psychological support of the wellness plan and uses registration ID to log in this part of the program. In step 2, the system generates a basic assessment according to the information provided in assessment questionnaire by the patient in communication with a set of inputs. The set of inputs provide data for generating personalized health content plan. The assessment is to determine issues in 6 areas: depression, anxiety, substance abuse, sleep, physical wellbeing and nutrition. This assessment helps system in choosing customized method and tips for setting goals, dealing with setbacks and effective goal settings. The tips for mental and wellness health content is preferably a large database of messages comprising video, audio and text data. Each element comprises a message. In particular, the message characterization comprises information on the treatment plans and psychological and/or wellness profiles for which the message is suitable or effective. In a preferred embodiment, the message characterization comprises tags indicating the effectiveness of the corresponding message for given treatment plan, psychological profile, and health profile parameters.

In step 3, the patient starts his/her support program and wellness plan. The patient receives information of mental health, mind-body connections and how the recovery plan works, the patient receives relaxation and breathing exercises, physical exercises and healthy habits and social supports which can help through this plan. The patient learns how to deal with depression, anxiety, substance abuse and how to confronting the problems. Patient receives information of physical, behavioral, emotional, mental, self and interpersonal problems and how to deal with them.

In relaxation and breathing exercises patient learn square breathing exercise, diaphragmatic or abdominal breathing exercise, progressive muscle relaxation, mindfulness relaxation and visualization exercise.

In physical wellbeing patient receive aerobic and low-impact exercises, nutritional improvement tips (elimination of caffeine, reduction of salt and sugar and processed foods and learn healthy eating habit) and sleep tips.

Patient can set up a toolbox, which provides daily trackers on mood, exercise, sleep and nutrition, and reminders for staying hydrated, eating healthy, about bedtime and waketime, physical exercises, relaxation exercises, mindfulness, 5-minute of journal, about vitamins and other supplements. The system also provides an emergency toolbox for patients to set up activities that help distract and alleviate depression, anxiety, and angry moods and emotions. Names and information's of contact persons who can be reached in crisis events are provided in this section.

In step 4, the system provides CBT (cognitive behavioral therapy) type of treatment to help patients understand thoughts and feelings that influence behaviors. The system will provide exercises for changing negative thinking pattern, negative self-talk and cognitive distortions (anxiety), general rules about self-talk and provide daily coping affirmations.

Patient can set up a toolbox which provides daily affirmations that can help person embody positive thinking and mindfulness, daily journaling reminders about irrational thoughts.

In step 5, patient system provides skills for DBT (Dialectical behavioral therapy) skills and how to live in the moment, cope healthy with stress and regulate emotions. The patient evaluates his/her distress tolerance using wise mind exercises and accepts techniques to cope with distress tolerance. The system provides a list of enjoyable activities, self-soothing exercises, improve the moment technique, provide a list of coping thoughts. Wellness toolbox of the system will provide quotes to improve the moment technique (listing life values and coping thoughts), distress tolerance log and daily relaxation and breathing and mindfulness exercise. Emergency toolbox of the system in this step provides techniques to distract from intense situations and grounding techniques, self-soothing strategies using the senses.

In step 6, the system provides exercises for mindfulness. The patient will learn being in the present moment, awareness without judgement, radical acceptance and how to use radical statements in life and strategies to deal with challenging situations exercise. The system provides an 8 week challenge mindfulness exercise:
Mindfulness exercise 1: Awareness in everyday activities
Mindfulness exercise 2A: Fully immersed in an activity using the senses
Mindfulness exercise 2B: Awareness of a piece of Toast using the senses
Mindfulness exercise 3: Awareness of time
Mindfulness exercise 4: Thousand thoughts a day
Mindfulness exercise 5: Putting emotions into words
Mindfulness exercise 6: Paying attention to the breath
The system provides effective meditation and relaxation exercises.

Step 7 is the self and interpersonal step in which the system provides tips and exercises for self-esteem, dealing with anger, motivation and goal-setting and being assertive. In this step, the patient is ready for change, set goals, visualize goals and put goals into action. The patient is ready for dealing with setbacks.

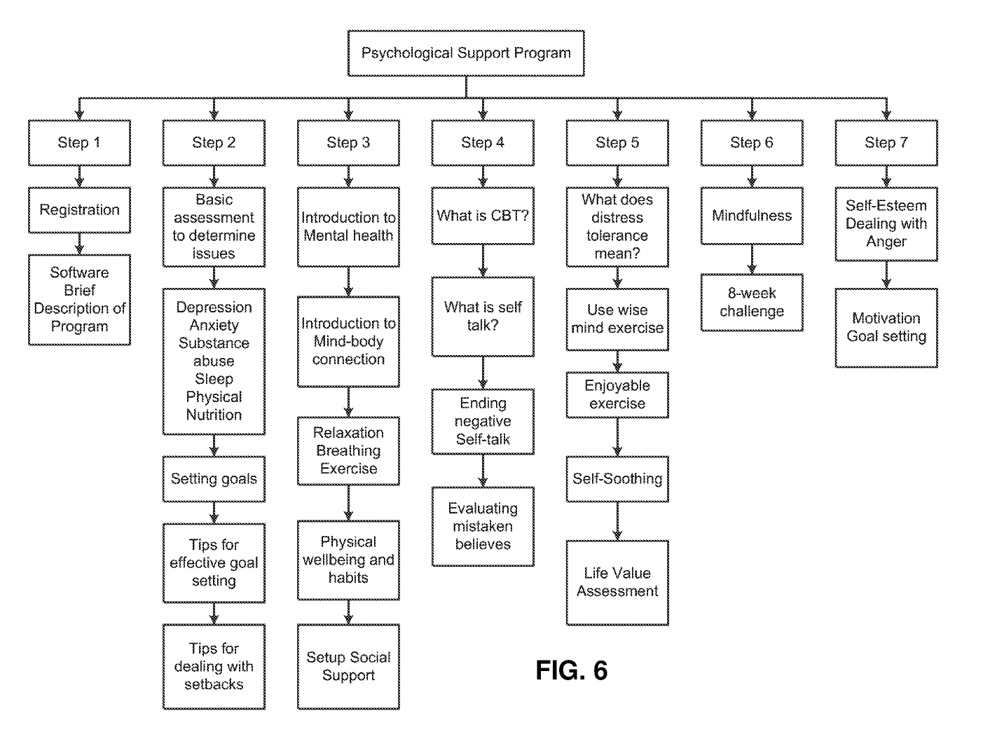

FIGS. 6A, 6B and 6C illustrate an exemplary notification that may be presented after the patient has logged in and set up the tool box. As shown, the pharmacological part of the system may provide a reminder for dosage of medical cannabis that patient should have to consume or the best way to consume. The method is based on the actual use data, a calculation based on an input of dosage of medical cannabis usage and opioid pills usage of the patient.

Daily encouraging posts based on psychological program of the invention and patients feedbacks will also be provided. It will be recognized that additional or alternative types of notifications may be provided. Patient can provide a respond to a Pain Treatment Survey by filing the Pain Diagram. In an embodiment of the present invention, a drawing of the human body, enabling the patient to illustrate the areas of pain can be provided. Questions can be posed concentrating on the highlighted areas-given. For example, the platform may generate a notification to take a specific amount of opioid and medical cannabis based on opioid reduction plan of the system generated for the patient. The system may provide a first notification to the patient to take 0.5 gram of medical cannabis and reduce %0 10 of the opioid drug (which will be calculated and provided by the health practitioner of the system). The system may also provide a second notification to the patient the best method to use the medical cannabis. The system sends notifications requesting the patient to rate their pain and health condition and provide notifications during the day. For example encouraging notifications, psychological exercises based on the reduction plan.

In some complicated cases for example when the patient is experiencing a severe pain out of the range of the opioid reduction plan, the system may notify a health care practitioner to initiate a consultation with the patient. The consultation may include a virtual consultation, an online video conference, an online audio conference, and or any other form of communication. Each of these types of content may be dynamically updated based on patients condition. For example, notifications may be dynamically changed as the patient improves in caring for his or her medical condition.

The foregoing is considered as illustrative only of the principles of the invention.

Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function and manner of operation, assembly and use are deemed readily apparent and obvious to those skilled in the art, and all equivalent relationships to those illustrated in the drawings and described in the specification are intended to be encompassed by the present invention

What is claimed is:

1) A computer-implemented method for an opioid dependency/addiction treatment for a patient using a communication server on a secure network and having a computer to store, process and transmit data to the patient, said method comprising the steps of:
a) collecting and storing a plurality of data regarding a plurality of pharmacological and psychological histories of opioid addiction of the patient;
b) presenting to the patient a plurality of selected-questions from a plurality of preset-questions stored in the computer;
c) receiving a set of answers to the plurality of selected-questions from the patient;
d) generating an opioid reduction plan by a professional healthcare practitioner based on said set of answers and, said opioid reduction plan comprising the steps of;
i) scheduling a daily opioid reduction dosage;
ii) scheduling a daily cannabis dosage as a replacement medication for said daily opioid reduction dosage;
iii) providing a set of posts related to said daily cannabis dosage and said daily opioid reduction dosage based on said opioid reduction plan;
iv) providing a set of daily motivative and encouraging posts to support said patient;
v) prescribing a daily set of exercises comprising of physical, behavioral, mental, emotional, self and interpersonal exercises;
vi) providing a set of cognitive behavioral therapy and mindfulness training proven to be effective for pain management, relief associated symptoms of anxiety, depression, or sleep disturbances, and increase function in valued social, vocational/avocational, creative, and recreational roles;
e) monitoring a response of said patient to said opioid reduction plan by repeating steps (b) and (c) for generating a second set of answers and comparing the second set of answers with said set of answers to assess a set of side effects comprising depression, anxiety, substance abuse, sleep, physical wellbeing and nutrition, and
f) changing said daily cannabis dosage to accommodate for said side effects and providing customized psychological support to manage said side effects.

2) The method of claim 1, wherein said daily cannabis dosage comprising of using 500 mg of a cannabis for every 10% reduction in the opioid.

3) The method of claim 1, wherein said daily cannabis dosage is between 0.5-3 g per day going up by increments of 0.5 to 1 gram at each dose change.

4) The method of claim 1, wherein said daily cannabis dosage is not to exceed 5 gram per day.

5) The method of claim 1, wherein said plurality of preset-questions is aimed at a plurality of biomarkers for:
a) rating a pain of said patient before and after said opioid reduction plan, wherein said pain is evaluated based on a pain intensity, a pain quality and a pain location;
b) identifying a set of clinical and psychological factors that increase and decrease said pain; and
c) reviewing of an effectiveness of the opioid to relieve the pain, a sleep, a stress, a working ability, a relationship, and a recreational activity.

6) The method of claim 5, wherein said pain intensity is evaluated through a numerical rating scale of 0 to 10 or a visual analog scale or verbal descriptor scales or facial distress image scales.

7) The method of claim 1, wherein said plurality of preset-questions comprising questions regarding the patient's psychological state comprising of an anxiety, a willingness to give-up, a potential memory impairment, and a reduced performance at work.

8) The method of claim 1, wherein said set of daily exercise provides daily exercises to improve and maintain patients quality of life, utilize techniques to better handling distress, monitor negative self-talk, mindfulness meditation, change the negative thoughts and bring awareness and change into negative self-talk, build and maintenance, self-esteem and motivation and goal-setting.

9) The method of claim 1, wherein said professional healthcare practitioner being any one of a physician, a nurse, a pain specialist, an addiction specialist, a psychologist or a psychiatrist.

10) The method of claim 1, wherein said method automatically alerts said patient of any acute effects of said opioid reduction plan based on said second set of answers, wherein said acute effects being a group consisting of perceptual distortions, cognitive impairment euphoria, anxiety, vasodilation, supine hypertension, postural hypotension, heightened sensory perception, drowsiness, incoordination, and increased appetite.

11) The method of claim 1, wherein said plurality of pharmacological and psychological histories of opioid addiction of the patient comprising of a type and dosage of said opioid, an addiction/dependency stage and a set of side effects comprising of a pain intensity, a pain quality and a pain location, a clinical and psychological factors that increase and decrease said pain.

12) The method of claim 1, wherein said plurality of preset-questions comprising questions about the patient's age, athletic activity, and demographic data.

13) A system for an opioid addiction treatment of a patient comprising of a pharmacological and psychological treatment, the system comprising:
a) a communication server for communicating with the patient over a secure network connection;
b) a data storage device for storing a plurality of data regarding pharmacological and psychological history of opioid addiction collected from the patient through a plurality of questions, and a plurality of preset-questions to be presented to the patient;
c) a processor configured to:
i) access said plurality of data,
ii) select a plurality of selected-questions from said plurality of preset-questions to be presented to the patient based on one or more of the plurality of data regarding pharmacological and psychological history of opioid addiction identified by the system,
iii) present the plurality of selected-questions by the system to the patient and receive a plurality of answers form the patient,
iv) generate an opioid reduction treatment plan, based on said data and said answers, wherein said plan comprises a daily dosage of medical cannabis as a replacement for opioid;
v) provide daily motivative reminders and posts to motivate and encourage said patient throughout said opioid addiction treatment, and
vi) monitor the patient based on said answers of said patient to said treatment plan and provide customized psychological support.

14) The system of claim 13, wherein said plurality of data further comprises of a clinical history of the patient.

15) The system of claim 13, wherein said plurality of data regarding pharmacological and psychological history of opioid addiction of the patient comprising:
a) a pain intensity and a pain location;
b) a set of clinical factors that increases and decreases said pain;
c) a set of clinical and psychological effects of said pain, and
d) a type and dosage of said opioid.

16) The system of claim 13, wherein said plurality of preset-questions comprises of questions about the patients pharmacological and psychological history.

17) The system of claim 13, wherein the set of psychological treatment comprises of daily physical and behavioral exercises.

18) The system of claim 13, wherein the daily dosage of medical cannabis comprises: using 500 mg of said cannabis for every 10% reduction in opioid, and wherein cannabis recommended daily amount is between 0.5-3 g per day going up by increments of 0.5 to 1 gram at each dose change.

19) The system of claim 13, wherein said communication server is an online platform, a website, or a mobile application.

20) The system of claim 13, wherein said system categorizes said patient into any one of
a) a pre-contemplation category, wherein the patient is not thinking of an opioid reduction, and wherein the system provides a motivational program;
b) a contemplation category, wherein the patient is considering potential benefits of opioid reduction, and wherein the system provides a motivational program;
c) a preparation category, wherein the patient is ready to begin opioid reduction, but is unsure, and wherein the system sets small, achievable goals to build confidence and provide support;
d) an action category, wherein the patient is committed to opioid reduction, and wherein the system provides a plan and monitors the patient, and
e) a maintenance category, wherein the patient may lose motivation over time, and wherein the system provides motivative and encouraging tips.
